(19) United States
(12) Patent Application Publication
(10) Pub. No.: US 2004/0251145 A1
(43) Pub. Date: Dec. 16, 2004

(54) High throughput screening (HTS) method and apparatus for monitoring ion channels

(76) Inventor: Janet Kay Robertson, Easton, PA (US)

Correspondence Address: BLANK ROME LLP, 600 NEW HAMPSHIRE AVENUE, N.W., WASHINGTON, DC, 20037, US

(21) Appl. No.: 10/782,943

(22) Filed: Feb. 23, 2004

Related U.S. Application Data
(60) Provisional application No. 60/448,490, filed on Feb. 21, 2003.

(51) Int. Cl. C25B 9/00; C25B 13/00; C12N 9/00; C12M 1/00

(52) U.S. Cl. 205/775; 204/403.01; 204/409; 205/792; 205/793.5

(57) ABSTRACT

The present invention relates to apparatus and method for high throughput determining and/or monitoring electrophysiological and fluorescence properties of ion channels or ion channel-containing structures, such as cell membranes, by establishing an configuration in which a cell membrane forms a highly resistive seal around an orifice, making it possible to determine and monitor a current flow through the cell membrane. The substrate can be part of an apparatus for simultaneously studying cell membranes using electrical and fluorescent techniques. The apparatus is formed of all transparent materials and preferably part of a multi-well plate.

This application claims priority to U.S. Provisional Patent Application No. 60/448,490, filed Feb. 21, 2003.

### FIELD OF THE INVENTION

The present invention relates to apparatus and method for high throughput determining and/or monitoring electrophysiological and fluorescence properties of ion channels or ion channel-containing structures, such as cell membranes, by establishing an configuration in which a cell membrane forms a highly resistive seal around an orifice, making it possible to determine and monitor a current flow through the cell membrane. The substrate can be part of an apparatus for simultaneously studying ion channels using electrical and fluorescent techniques.

### BACKGROUND OF THE INVENTION

The passage of ions across biological membranes to transduce a biological effect is exquisitely regulated. Ion channels, which are membrane-associated proteins, are important contributors to this regulation process. Ion channels are prevalent in the body and are necessary for many physiological functions including the beating of the heart, voluntary muscle contraction and neuronal signaling. They are also found in the linings of blood vessels allowing for physiological regulation of blood pressure and in the pancreas for control of insulin release. As such, the study of such channels is a very diverse and prolific area encompassing basic academic research as well as biotech and pharmaceutical research. Experiments on ion channels are typically performed on cell lines which endogenously express the ion channel of interest (“native channels”) as well as on recombinant expression systems such as the Xenopus Oocyte or mammalian cell lines (e.g. CHO, HEK etc.) where the channels are inserted by well-known transfection techniques. Electrophysiology is also performed on isolated cell membranes or vesicles as well as in synthetic membranes where solubilized channels are reconstituted into a manufactured membrane. These channels are able to respond to a variety of stimuli, including a change of electrical potential across the plasma membrane. To study the effect of pharmaceuticals on voltage-gated ion channels, it is necessary to apply a voltage across the cellular membrane while observing the operation of the channels. At present, this can only be achieved by the use of microelectrodes, which is an invasive and labor-intensive process.

Ion channel are perhaps the most important, poorly assayed target class. Yet, the treatment of such diseases as Glaucoma and Cancer may require the discovery of ion channel modulating or blocking pharmaceuticals. Therefore, a need exists for a new invention which allows the effect of a stimulus such as a pharmacological agent or a toxin on an ion channel to be rapidly assessed.

The “gold-standard” for testing ion channels is the patch clamp technique. Conventional patch clamping relies on maintaining (clamping) a constant voltage across a cell membrane via a glass micropipette (patch pipette) with a 1-μm to 2-μm-diameter tip opening. A patch pipette is fabricated by heating the center of a glass capillary while pulling the two ends in opposite directions. Heating softens the glass as pulling stretches and tapers the soften capillary until it separates into two pieces. Each piece becomes a patch pipette, which is then filled with a salt solution. A silver wire with a silver-chloride coating is inserted into the pipette completing fabrication.

The pipette tip is then carefully positioned on the surface of a living cell. Gentle suction is applied through the pipette to draw a portion (a patch) of the cell membrane into the tip. The pipette rim and the membrane patch form a mechanically and electrically tight junction, referred to as a “giga-seal” with an electrical resistance measured in the range of 10 GΩ to 100 GΩ (1 GΩ=10⁹Ω). Without damaging the gigaseal, the membrane patch is then broken open by a pulse of suction, a voltage pulse, or an applied chemical which creates physical pores in the membrane, to create a cell interior contiguous with the pipette solution. Using a recording electrode (typically a Ag/AgCl wire) inside the pipette and a reference electrode in the extracellular solution, the cell membrane potential is maintained constant at a preset voltage. Both electrodes are connected to a patch clamp amplifier that supplies the current necessary to maintain the cell membrane potential at the command voltage. This current, which can be measured and is equivalent to the net ion flow through the cell membrane, reveals valuable information about the functioning of the ion channels and cell signaling. The technique was invented by Sakmann and Neher. Both scientists shared the 1991 Nobel Prize for their invention. This technique can also be applied to portions of a cell membrane or synthetic membranes.

Conventional patch pipettes have served a remarkable mission in cell physiology, particularly as a tool for recording electrical activity from cells or delivering biochemical reagents of interest into cells. However, the patch clamp technique is labor and skill intensive resulting in high cost per experiment and low throughput. Therefore, patch clamping is not currently used in the high throughput screening of drug candidates where thousands of different conditions (e.g. chemical stimuli) must be tested each day. Thus, a need exists for an invention which automates the patch clamp technique. Furthermore, it is also desirable to combine the patch clamp technique with fluorescence technology since fluorescence can be used to gather different information than that which is gathered electrically using only traditional patch clamp methods. For example, fluorescence technology can be used to gather information on the movement of molecules within the cell in response to applied stimuli.

Several companies are attempting to automate the patch clamp technique. U.S. Pat. No. 6,488,829 to Schroeder et al. discloses an apparatus which uses a thin, preferably layered substrate having a properly sized hole, on the order of a few microns in diameter, allowing a cell or biological membrane to be maneuvered by fluid flow through the hole independent of direct human intervention, to form an insulative seal between the cell and the hole. The apparatus, thereby, eliminates the use of a microscope and micromanipulating arm.

WO 99/66329 to Owen et al. discloses a substrate with perforations arranged in wells and electrodes provided on each side of the substrate. The substrate is made by perforating a silicon substrate with a laser and may be coated with anti-adhesive material on the surface. The substrate is adapted to establish high resistive seals with cells by positioning the cells on the perforations using suction creating a liquid flow through the perforations, providing the anti-adhesion layer surrounding the perforations, or by guiding the cells electrically. The cells can be permeabilised by EM fields or chemical methods in order to provide a whole-cell measuring configuration. All perforations, and hence all measurable cells, in a well share one working electrode and one reference electrode, hence measurements on individual cells can not be performed.

U.S. Pat. No. 6,682,649 to Petersen et al. discloses a substrate and a method for obtaining an electrophysiological measuring configuration in which a cell forms a giga-seal around a measuring electrode making it suitable for determining and monitoring a current flow through the cell membrane. Substrate has a plurality or an array of measuring sites with integrated measuring and reference electrodes formed by wafer processing technology. However, the substrate is not suitable for simultaneous patch clamping and fluorescence studies because it does not use all transparent materials in the construction of the substrate.

### SUMMARY OF THE INVENTION

The present invention relates to an apparatus and method for the high throughput screening (HTS) and/or monitoring of the electrophysiological properties of ion channels or ion channel-containing structures, such as cell membranes, by establishing a configuration in which a cell membrane forms a highly resistive seal around an orifice making it possible to determine and monitor a current flow through the cell membrane. If transparent electrodes, a transparent substrate and a transparent insulator are used, this invention can also include the monitoring of the cell or ion channel containing structure, using fluorescence technology. Fluorescence is a highly developed optical method for interrogating the response of a cell to an applied stimulus whether that stimulus be electrical or pharmacological or another type of stimulus. Fluorescence monitoring includes the application of a dye to the fluid either inside or outside of the cell. The dye absorbs light of one frequency and then re-emits the light at a different frequency when a characteristic event occurs, such as when a certain chemical is present. Many different dyes are available which respond to many different characteristic events. Multiple fluorescing dyes can be added to measure a variety of events simultaneously. This invention allows the response of the ion channel containing structure to be monitored electrically, monitored optically using fluorescence, or monitored both electrically and optically simultaneously.

The apparatus of the present invention comprises a small (about 0.5-2μm) orifice in an insulation layer disposed over an electrode. The electrode, disposed under the insulation and on top of a substrate, is exposed to the extra cellular solution only within the area of the small orifice. Otherwise, the electrode is insulated from the extra cellular solution elsewhere. Cells cultured on the insulation grow over the orifice. The orifice is sufficiently smaller than a cell or ion containing structure diameter so that a single cell or ion containing structure covers the orifice; and cell membrane forms a seal to the underlying insulation layer with a seal resistance between 1 MΩ and 1 GΩ. Thus, the cells, when they grow, position themselves over the transparent electrode. Cells which are incorrectly positioned can be sensed electrically and electronically eliminated from the test. Coatings which facilitate cell growth and/or adhesion can be used to encourage cell growth and/or adhesion in the areas immediately adjacent to the orifice.

Many types of HTS which do not involve an applied electrical stimulus are conducted using micro well plates. These plates typically have 96, 384, or 1536 wells. They are typically manufactured using injection molding and are often made of polystyrene. The plates are constructed such that the bottom of each well is transparent. This facilitates the use of fluorescence techniques with light entering and leaving the plate through the transparent substrate. The plates are “read” (the optical response of the cells is measured) by a large expensive piece of equipment called a plate reader. Most micro well plates manufactured today are made to a set of ANSI standards so that they can be used with most plate readers.

If this invention uses transparent electrodes, a transparent substrate and a transparent insulator, the invention can be incorporated into a standard micro well plate or into a device with the same footprint as a standard micro well plate so that the plate is compatible with most plate readers thus facilitating its use with fluorescence technology. Thus, the device can be used with HTS machines such as the FLIPR and the VIPR.

This invention can also be combined with patch clamp electronics. These can be external to the device or integrated into the micro well plate such that a portion of the device on which the cells were cultured was disposable and the remainder containing the patch clamp electronics was reusable. Since multiple orifices can be manufactured in one well, with multiple electrodes, the invention described can set the membrane potential of many cells cultured in a single well simultaneously. One small orifice in each well can be used as a reference electrode. This electrode is exposed to the extracellular solution due to the lack of a cell covering it. Thus, the area immediately adjacent to the reference orifice is coated with a substance that inhibits or discourages cell growth and/or adhesion.

Methods for using the apparatus of the present invention is also provided.

The advantages of the present system includes: 1. Simultaneous studies of multiple cells; 2) facilitation the use of fluorescence and high content screening due to transparent apparatus; 3) reduced cost; 4) no positioning of the cell over the orifice is required; 5) cells without a specified seal resistance can be eliminated from the test by individually testing each orifice prior to performing the experiment; 6) facilitation of the study of signaling between cells; 7) microfluidic channels can also be embedded in the apparatus to allow suction to be applied to increase the seal resistance; 8) a centrifuge or suction can be used to force a small portion of the cellular membrane into the orifice; 9) if two electrodes are positioned beneath the cell, one electrode can be used to supply current and the other to monitor the cell membrane potential.

### BRIEF DESCRIPTION OF THE DRAWINGS

FIG. 1 shows an embodiment of the present invention: A) a cross-section view; and b) a top view.

FIG. 2 shows an embodiment of the reference electrode.

FIG. 3 has been drawn to scale and shows the relationship between the size of a 1 μm orifice and a cell with a 20 μm diameter centered on top of the opening.

FIG. 4 shows the plurality of the orifices and a set apart reference electrode.

FIG. 5 shows the present invention in a multi-well plate.

FIG. 6 shows an embodiment of the present invention where ridges are formed on the opening lip of the orifice.

FIG. 7, shows an embodiment of the present invention where the well is tapered and microfluidics channels are incorporated into the layered structure.

### DETAILED DESCRIPTION OF THE PREFERRED EMBODIMENT

The present invention provides apparatus and method for performing multiplexed patch clamp technique. The invention preferably has a plurality of orifices, preferably about 100 to 2000, more preferably about 500 to 1500, and most preferably about 1000, where each orifice has a diameter of about 1 to 5 μm, and associates with at least one electrode. Preferably, each electrode is electrically isolated from every other electrode.

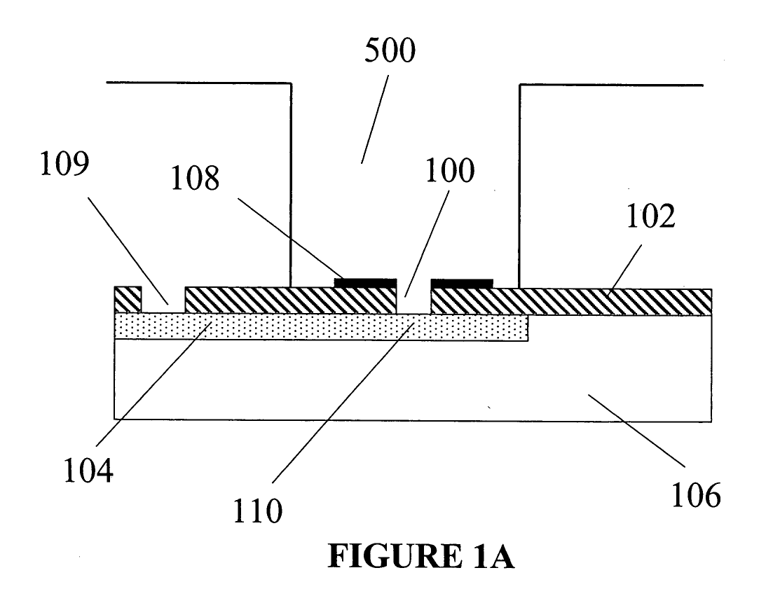

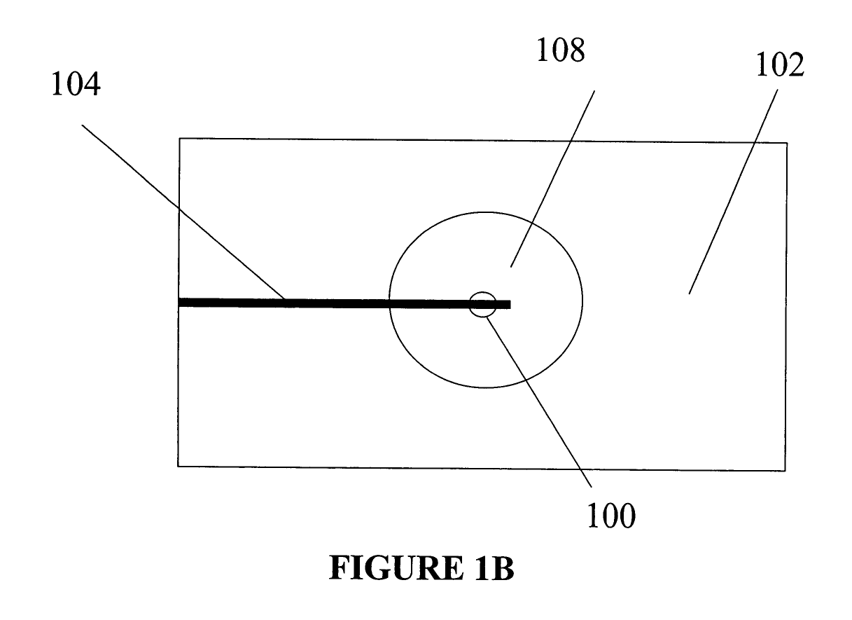

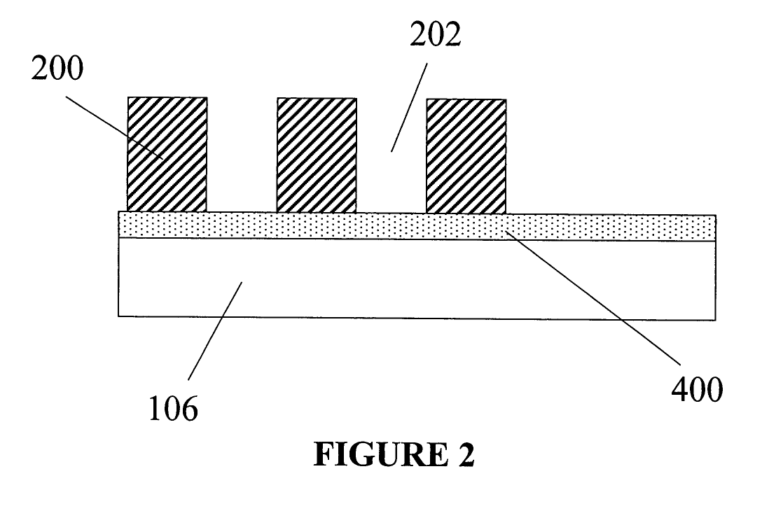

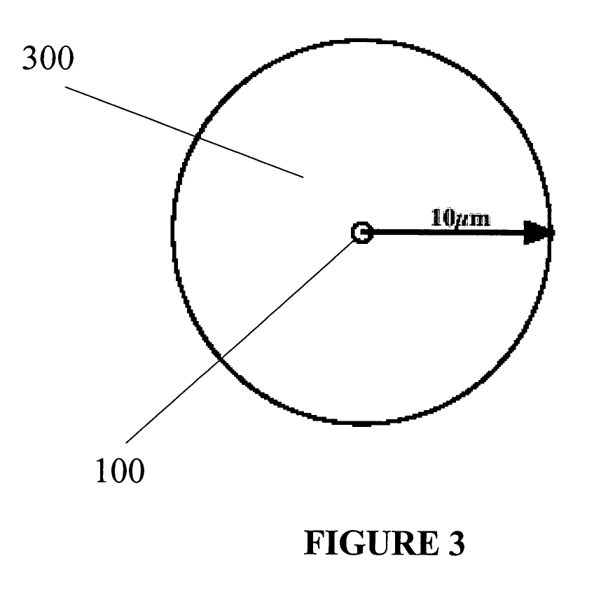

FIGS. 1 and 2 depict an orifice of the present invention. The apparatus is constructed of a layered structure. As shown in FIGS. 1 and 2, the orifice 100 is formed by creating an opening in insulator 102. On a portion of substrate 106, electrode 104 is positioned between insulator 102 and substrate 106. Orifice 100 is created only where electrode 104 is between insulator 102 and substrate 106 such that a well is formed with sidewalls formed from insulator 102 and a bottom surface formed from electrode 104.

The substrate 106 having orifices 100 formed thereon with measuring electrode(s) 104 can be designed in a number of ways. The orifices are adapted to hold an ion channel-containing structure, such as a cell, in that the surface material at the site is well suited for creating a seal with the cell (or structure) membrane. As such, the materials for insulator 102 include, but are not limited to, silicon, plastics, pure silica and other glasses such as quarts, Pyrex, or silica doped with one or more dopants such as Be, Mg, Ca, B, Al, Ga, Ge, N, P, As and oxides from any of these.

Substrate 106 can be made of silicon, plastics, pure silica and other glasses such as quarts, Pyrex, or silica doped with one or more dopants such as Be, Mg, Ca, B, Al, Ga, Ge, N, P, As and oxides from any of these. Glass is presently the preferred substrate material. Most preferably, all materials used for making the present invention are transparent for use in conjunction with fluorescent assays. Transparent as used herein refers to light transmission, preferably greater than 50% tranmissive, more preferably greater than 75% trnasmissive, and most preferably greater than 80% transmissive, over the range of emission and absorption of the fluorescent molecule used.

Electrode 102 are formed on the surface of the substrate by first depositing a layer of conducting material on the substrate 106. Deposition of materials on the substrate, and on other surfaces throughout the description, can be made using one of several deposition techniques, such as Physical Vapour Deposition (PVD) which includes applying of material from a vapour phase, sputtering, and laser ablating; Chemical Vapour Deposition (CVD) techniques which include atmospheric pressure chemical vapour deposition (APCVD), low pressure chemical vapour deposition (LPCVD), plasma enhanced chemical vapour deposition (PECVD), and photo enhanced chemical vapour deposition; as well as spin coating and growth techniques. Secondly, the individual wires are defined in a photolithography step. And thirdly, conducting material not being a part of the wires is removed by etching. Alternately, a liftoff process or shadow mask process can be used. If a liftoff process is used photoresist is first patterned on the wafer such that the photoresist is removed where ever metal is desired. The metal is then deposited using one of the techniques previously described. The photoresist is then dissolved causing the undesired metal on top of the photoresist to “liftoff” of the surface. A shadow mask can also be used to selectively deposit metal through holes in a thin metal plate placed between the wafer and the deposition source.

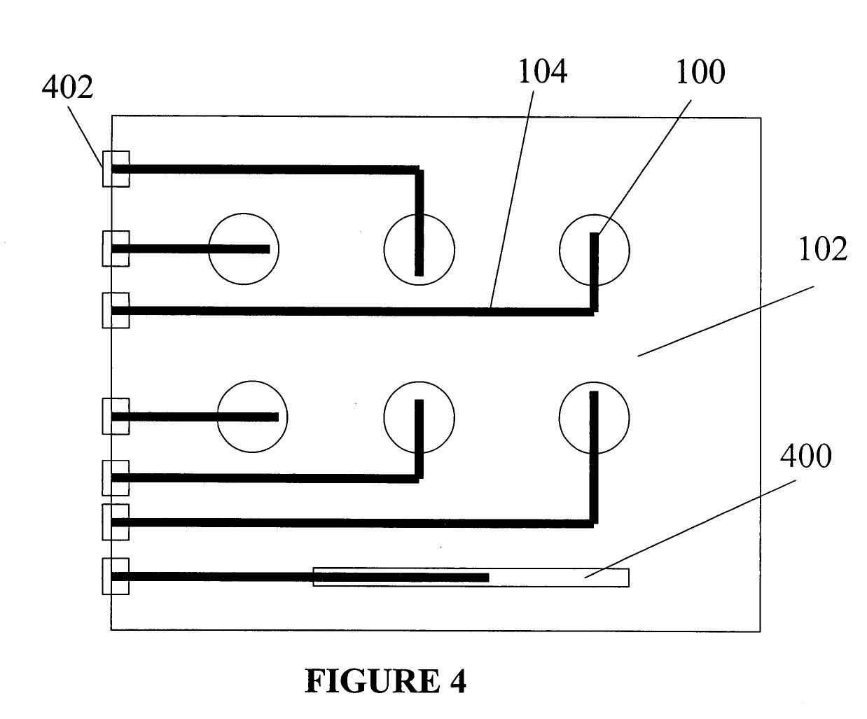

The wires are preferably defined so that one part of the wires forms a line of contact pads 402, shown in FIG. 4, for attachment to other electronic device(s) associated with patch clamping, while another part forms an array of measuring electrodes and one or more reference electrodes. The array of electrodes is not necessarily an ordered pattern. The contact pad and electrodes are portions of electrode 104. The conducting material consists of metals, preferably a transparent metal such as indium tin oxide (ITO). Each electrode is preferably electrically insulated from the other electrodes. Alternately, however, groups of electrodes can be connected together to minimize the total number of leads brought out to the contact pads 402. Ti/Pt or another low resistance metal is used to connect the electrodes to the contact pads 402. However, because Ti/Pt is opaque, it is preferably used outside of the detection area so that fluorescence detection through the substrate can be used to monitor cellular response.

Electrodes 104 are electrically insulated from the extracellular solution by insulator 102. Insulator 102 is formed by first depositing the insulator using physical or chemical vapor deposition, or spin coating. Photolithography combined with wet or dry etching is then used to selectively remove insulator 102 forming orifice 100.

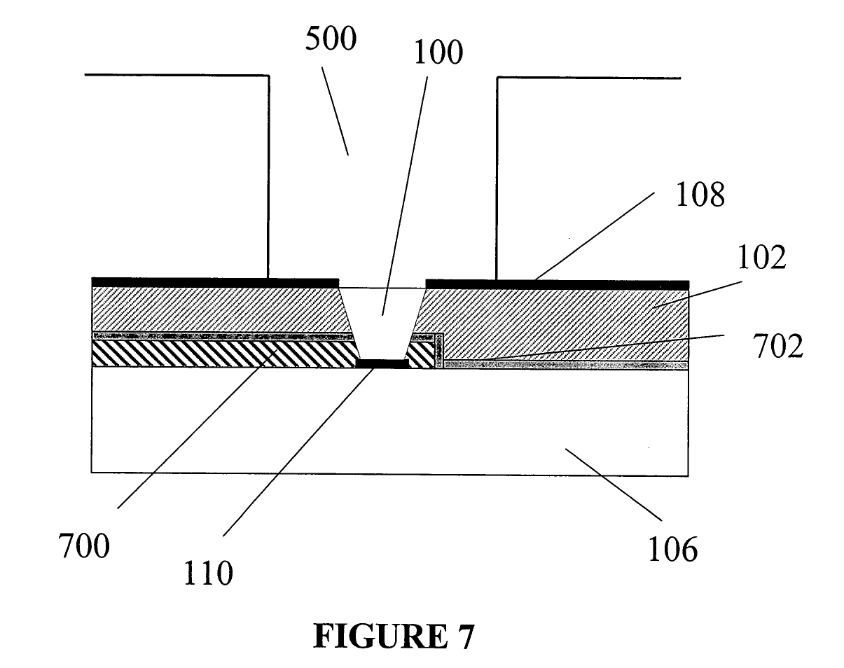

In a preferred embodiment, the area adjacent to and surrounding orifice 100 is coated with a substance 108 to promote cell growth and/or adhesion. These substance can be, but are not limited to, inorganic substances, such as SiO2, and divalent cations (Ca²⁺); and/or organic substances, such as polylysine and collagen. Coating 108 encourages cell growth over the orifice or cell adhesion to the orifice. Coating 108 enhances the ability of the cell to form an electrically insulating seal with a resistance of 1 MΩ to 1 GΩ. In an embodiment, the whole surface can be coated with substance 108 to promote cell growth and/or adhesion (see FIG. 7).

Further, the ability of the cell to form an electrically insulating seal can be enhanced by altering the shape of the orifice. In one embodiment, the orifice is conical with smoothly tapered side-walls (FIG. 7). The side-walls are tapered to provide a smooth transition from the exterior of the orifice to the interior of the orifice. This smooth transition increases the total surface area with which the exterior of the cell membrane contacts the interior of the orifice. For example, the opening of orifice 100 can be about 3 μm tapering to about 1 μm at electrode 110. In any event, the diameter of the opening is no larger than the diameter of the cell being studied, preferably no larger than half the diameter of the cell being studied.

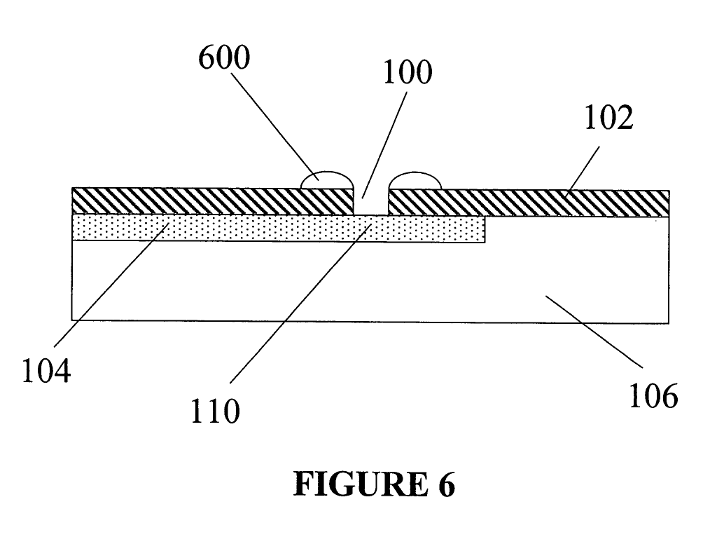

In another embodiment, ridge 600 is formed on the lip of the orifice (FIG. 6). This ridge 600 helps seal the cell against the orifice 100. The ridge most preferably has a height of about 1000-2000 Å above the lip of the orifice 100, and formed of silica, preferably ultra-pure silica. The ridge 600 can also be coated with a substance to encourage cell growth and/or adhesion as discussed above.

In yet another embodiment, the apparatus includes at least a subsurface microfluidic channel 700 in fluid communication with the orifice 100 (FIG. 7). In this configuration, it is preferred that the electrode(s) 110 of each orifice 100 are at about 90 degrees to each other. In making the microfluidic channels, the ITO electrode 110 is deposited and patterned first. Then a photoresist is deposited and patterned. This photoresist layer will form the channel 700 when removed later in the process. Next a thin 0.5 μm layer of parylene 702 is deposited. On top of this is spun polyimide (insulating layer 102, about 1-3 μm thick). Finally a layer of 1000 Å to 5000 Å of SiO₂ (substance 108 that promotes cell growth and/or adhesion) is deposited on the polyimide. This last layer is photopatterned using photo resist and either wet or dry etching. Then without removing the photoresist the device is placed in an O₂ plasma and the polyimide and parylene are etched. Precise stopping on the photoresist layer is not required. The etch can proceed into the 0.5-1 ∞m layer of photoresist if desired. Then the photoresist, both that in channel 700 and that on top of the SiO2 layer are removed in acetone. The tapered orifice is formed by adjusting the plasma etch parameters.

The reference electrode can be specifically designed to be used. FIG. 4 shows an embodiment in which the reference electrode 400 is set apart from and has a different configuration than the orifices. Alternatively, Any orifice which is not completely covered by a cell and is in low resistance contact with the extracellular solution can be used as a reference electrode. In a preferred embodiment, shown in FIG. 2, the reference electrode is designed to be free of cells or ion-channel containing structures attached thereon. This can be engineered in several ways. One way is to coat the electrode and/or areas immediately adjacent to the electrode with substances that discourage cell growth and/or attachment. These substances can be, but is not limited to, polydimethylsiloxanes (PDMS) and/or teflon. In certain embodiments, however, inhibition of cell growth and attachment is not necessary because the small gaps between the cells provides adequate contact between the reference electrode and the extracellular solution to facilitate proper operation of the system. This, of course, depends on the growth condition and type of cell being studied. For example, if the cells grow to 100% confluency, then inhibition of cell attachment to the reference electrode is required. Further, inhibition of cell attachment to the reference electrode is also important for use in conjunction with fluorescence detection, especially where the fluorescence detection cannot differentiate cells that grows on top of the reference electrode from those in solution.

Moreover, surface topography of the electrode can also be engineered to discourage cell growth and/or attachment, for example as shown in FIG. 2, patterning tall islands 200 of an appropriate material. The separation 202 between adjacent islands is less than half the diameter of a cell to discourage cell growth between the islands 200. These islands 200, if of sufficient height (about ¼ to ¾ the diameter of a cell), and of small diameter (less than about ¼ the diameter of a cell) also mechanically discourage the growth of cells on top of the islands. Tall islands 200 can be formed from a photopatternable material such as polyimide.

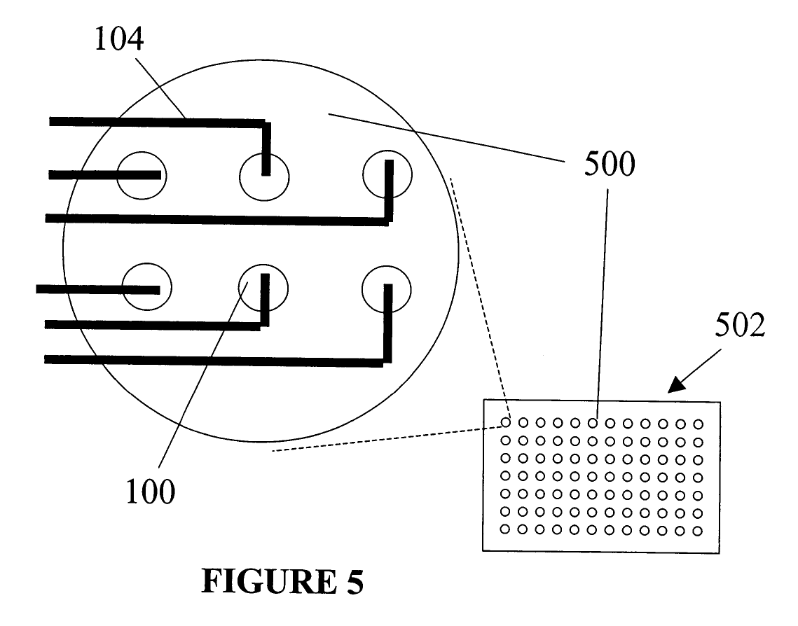

In an embodiment, the present invention is a modified multi-well plate 502 as shown in FIG. 5. The multi-well plate can have 96, 384, 1536 wells, or any other systems available in the art. Each well 500 contains a plurality of orifices 100, preferably up to about 1000. Each orifice 100 are constructed as shown previously in FIGS. 1 and/or 7, having the bottom of the multi-well plate forming the insulator layer 102. Beneath each orifice 100 are one or two transparent electrodes 104. A transparent metal such as ITO can be used to form these electrodes. The bottom surface of the well immediately adjacent to the orifices is coated with a substance to promote cell adhesion. The remainder of the surface is either not coated or coated with a substance which discourages cell adhesion. All of the wells can be used or only a few, perhaps one row of 8 (in a 96-well plate), can be used. The remainder of the plate area would then be allocated to the controlling electronics. In this case, the 8 wells would drop into a larger reusable device with the dimensions of a plate. The multi-well of the present invention, preferably has the same footprint as that of the standard plate to make it compatible with HTS plate readers, such as FLIPR and VIPR. If the footprint is different than that of the standard plate, adaptors can be manufactured to adapt the present multi-well plate to be used with standard equipments.

The present invention is preferably used in multiplexed measurement of cellular responses to external stimuli. Particularly, the present invention is most useful in screening compounds for effects on ion channels. Determining whether a test compound has an effect on ion channels involves contacting the cell or ion channel containing structure with the test compound and assaying for the flow of current or ions, or for other indicators of ion transport across the membrane. The flow of current or ion, or ion transport is then compared to a control, e.g., the same reaction in the absence of the test compound or in the presence of a known effector compound. Because of the use of multi-well plate, each well containing a different test compound, the present invention can simultaneously test and record results for a plurality of different test compounds.

In operation, a cell or ion containing structure, typically a cell membrane containing ion channels, is grown over the orifice 100 forming an electrically insulative seal of about 1 MΩ to 1 GΩ. The insulative seal is important to ensure that the current measured is the result of ion transport accoss the cell membrane and not of leakages of ions around the seal. The seal can be ensured by compounds that encourage adhesion as disclosed above, or by centrifugation to press the cells against the orifice 100. For example, spin speeds of up to 1000 rpm could be used to apply forces in the range of 10-20 μN. In using centrifugation, the aspect ratio of the orifice cylinder (the ratio of the depth of the orifice to the diameter of the orifice) can be varied to achieve a proper seal. It is preferred that this aspect ratio is about 3 (e.g., depth=3 μm and diameter=1 ∞m) to allow the cell to deform slightly into the orifice under the force of centrifugation.

The formation of the electrical resistive seal enables the measurement system to detect very small physiological membrane currents, (e.g. 10⁻¹² A). In addition, by perforating a portion of the cell membrane either electrically or chemically, it possible to control the voltage (voltage clamp) or current (current clamp) across the remaining intact portion of the cell membrane. This greatly enhances the utility of the technique for making physiological measurements of ion channel/transporter activity since quite often this activity is transmembrane voltage dependent. By being able to control the trans-membrane voltage (or current), it is possible to stimulate or deactivate ion channels or transporters with great precision and as such greatly enhance the ability to study complex drug interactions.

The interior of the cell can be accessed by a variety of methods. One technique locally destroys the cell membrane over the 1 μm opening by voltage pulses of sufficient strength and duration such that the membrane directly over the orifice physically breaks down. This is commonly referred to as “zapping” and is a well-known technique in the field. Another technique utilized to electrically permeabilize the membrane is through the use of certain antiobiotics such as Nystatin and Amphotericin B. These chemicals work by forming chemical pores in the cell membrane that are permeable to monovalent ions such as chloride. Because chloride is the current carrying ion for the commonly used Ag/AgCl electrode, these antiobiotics can produce a low resistance electrical access to the interior of the cell. The advantage of the chemical technique is that the membrane patch remains intact such that larger intracellular molecules remain inside the cell and are not flushed out by the pipette solution as with the zapping technique. The use of chemicals to electrically permeabilize the membrane is also a commonly used technique in the field and is referred to as a “perforated patch.” The “zapping” technique is preferred for the present invention. The cellular membrane can also be locally destroyed by applying a pulse of suction. Thus, if an subsurface channel is included this channel can be used to apply suction to the orifice, draw in a portion of the cell membrane, and in a controlled fashion break open the cellular membrane. If each orifice 100 is associated with two electrodes 104, one is used to sense the membrane voltage and the second to supply current (either positive or negative) to maintain the membrane voltage at the desired value.

Once the interior of the cell is accessed, the cell can be exposed to a test compound; and the current flow across the cell membrane can be determined. Besides measuring currently flow, the present invention can also be used in conjunction with other assays using fluorescent reporter molecules. Fluorescent assays are well known in the art. Preferably, the optical detection method in monitoring the activity of an ion channel involves fluorescence resonance energy transfer (FRET). Methods of FRET have been described in U.S. Pat. No. 5,661,035 to Tsien et al., which is incorporated herein by reference. In another preferred embodiment, in order to optically monitor biological activities such as the secretion or absorption of a biological molecule by the cell, plasma membrane rearrangement, intracellular rearrangement, cellular respiration, apoptosis, and gene transcription, changes in refractive index of the sample, a fluorescent of luminescent protein, or a fluorescently labeled small molecule precursor to a secreted substance, or fluorescently labeled nucleic acid molecule such as DNA or RNA, can be used as the optically detectable marker.

The invention has been disclosed broadly and illustrated in reference to representative embodiments described above. Those skilled in the art will recognize that various modifications can be made to the present invention without departing from the spirit and scope thereof.

What is claimed is:

1. An apparatus for the performing multiplexed patch clamping comprising:
an insulating layer having a plurality of orifices therethrough;
at least one electrode electrically connected with each orifice; and
a substrate supporting said insulating layer and said electrodes, wherein said insulating layer, said electrodes, and said substrate are transparent.

2. The apparatus of claim, 1 wherein each of said plurality of orifices has a diameter of about 0.5-5 μm.

3. The apparatus of claim 1, wherein said electrodes is a ITO electrode.

4. The apparatus of claim 1, wherein each electrode is electrically isolated from the other electrodes.

5. The apparatus of claim 1, wherein a part of each of said electrode is electrically connected to an orifice.

6. The apparatus of claim 1, wherein each orifice is electrically connected to two electrodes.

7. The apparatus of claim 6, wherein one of the two electrodes is a sensing electrode and the other electrode is used to deliver an electrical signal to the orifice.

8. The apparatus of claim 1, wherein an area adjacent to each orifice is coated with a substance to promote cell growth and/or adhesion.

9. The apparatus of claim 8, wherein the substance to promote cell growth and/or adhesion is silica (SiO₂), polylysine, and/or collagen.

10. The apparatus of claim 1, further containing a reference electrode.

11. The apparatus of claim 10, wherein said reference electrode is modified to inhibit cell growth and/or adhesion thereon.

12. The apparatus of claim 11, wherein the modification comprises coating said reference electrode with a substance that inhibit cell growth and/or adhesion.

13. The apparatus of claim 11, wherein the modification comprises altering the surface topography of said reference electrode.

14. The apparatus of claim 11, wherein the modification comprises depositing islands of insulating material on said electrode, wherein the separation between adjacent islands is less than half the diameter of a cell.

15. The apparatus of claim 11, wherein the modification comprises depositing islands of insulating material on said reference electrode, wherein the island has a height from the surface of said reference electrode of about ¼ to ¾ the diameter of a cell and a diameter of less than about ¼ the diameter of the cell.

16. The apparatus of claim 10, wherein said reference electrode is sandwiched between said insulating layer and said substrate.

17. The apparatus of claim 1, wherein said electrodes are sandwiched between said insulating layer and said substrate.

18. The apparatus of claim 1, wherein said insulating layer is constructed of silicon, plastics, pure silica or other glasses.

19. The apparatus of claim 1, wherein said substrate is constructed of silicon, plastics, pure silica and other glasses.

20. The apparatus of claim 1, wherein each orifice is fluidly connected to at least one microfluidic channel.

21. The apparatus of claim 20, wherein the at least one microfluidic channel is located between the insulating layer and the substrate.

22. The apparatus of claim 1, wherein the substrate defines the bottom of a well of a multi-well plate.

23. A method for performing multiplexed patch clamping and fluorescence assays comprising the steps of:
providing the apparatus of claim 1;
attaching a cell on each of said plurality of orifices to form an electrically resistive seal of about 1MΩ to 1 GΩ around the orifice; and
performing patch clamping and fluorescence assays on the cell.

24. The method of claim 23, wherein the attaching step is accomplished by growing the cell on the orifice or allowing the cell to migrate over the orifice under cellular motility.

25. The method of claim 23, wherein the performing step comprises
exposing the cell to a test compound; and
measuring a current across the cell membrane and/or a fluorescent signal indicating cellular activity in response to the test compound.

26. The method of claim, 23 wherein each of said plurality of orifices has a diameter of about 0.5-5 μm.

27. The method of claim 23, wherein said electrodes is a ITO electrode.

28. The method of claim 23, wherein each electrode is electrically isolated from the other electrodes.

29. The method of claim 23, wherein a part of each of said electrode is electrically connected to an orifice.

30. The method of claim 23, wherein each orifice is electrically connected to two electrodes.

31. The method of claim 30, wherein one of the two electrodes is a sensing electrode and the other electrode is used to deliver an electrical signal to the orifice.

32. The method of claim 23, wherein an area adjacent to each orifice is coated with a substance to promote cell growth and/or adhesion.

33. The method of claim 32, wherein the substance to promote cell growth and/or adhesion is silica (SiO₂), polylysine, and/or collagen.

34. The method of claim 23, further containing a reference electrode.

35. The method of claim 34, wherein said reference electrode is modified to inhibit cell growth and/or adhesion thereon.

36. The method of claim 35, wherein the modification comprises coating said reference electrode with a substance that inhibit cell growth and/or adhesion.

37. The method of claim 35, wherein the modification comprises altering the surface topography of said reference electrode.

38. The method of claim 35, wherein the modification comprises depositing islands of insulating material on said electrode, wherein the separation between adjacent islands is less than half the diameter of a cell.

39. The method of claim 35, wherein the modification comprises depositing islands of insulating material on said reference electrode, wherein the island has a height from the surface of said reference electrode of about ¼ to ¾ the diameter of a cell and a diameter of less than about ¼ the diameter of the cell.

40. The method of claim 34, wherein said reference electrode is sandwiched between said insulating layer and said substrate.

41. The method of claim 23, wherein said electrodes are sandwiched between said insulating layer and said substrate.

42. The method of claim 23, wherein said insulating layer is constructed of silicon, plastics, pure silica or other glasses.

43. The method of claim 23, wherein said substrate is constructed of silicon, plastics, pure silica and other glasses.

44. The method of claim 23, wherein each orifice is fluidly connected to at least one microfluidic channel.

45. The method of claim 44, wherein the at least one microfluidic channel is located between the insulating layer and the substrate.

46. The method of claim 23, wherein the substrate defines the bottom of a well of a multi-well plate.

47. The method of claim 23, further comprising the step of centrifuging the apparatus of claim 1 to press the cell against the orifice to ensure an electrically insulative seal of about 1MΩ to 1 GΩ.
